(19) United States
(12) Patent Application Publication
(10) Pub. No.: US 2021/0393711 A1
(43) Pub. Date: Dec. 23, 2021

(54) BACTERIA EXPRESSING SINGLE CHAIN ANTIBODIES AGAINST TOLL-LIKE RECEPTORS

(71) Applicant: YISSUM RESEARCH DEVELOPMENT COMPANY OF THE HEBREW UNIVERSITY OF JERUSALEM LTD., Jerusalem (IL)

(72) Inventors: Gabriel NUSSBAUM, Jerusalem (IL); Carsten KIRSHNING, Essen (DE)

(21) Appl. No.: 17/279,136

(22) PCT Filed: Oct. 10, 2019
(86) PCT No.: PCT/IL2019/051107

§ 371 Date: Mar. 24, 2021

Related U.S. Application Data
(60) Provisional application No. 62/744,160, filed on Oct. 11, 2018.

(51) Int. Cl. A61K 35/747; C12N 1/20; C07K 16/28; A61P 1/00; A61P 35/00

(52) U.S. Cl. CPC A61K 35/747; C12N 1/20; A61P 35/00; A61P 1/00; C07K 16/28

(57) ABSTRACT

The present invention provides transgenic bacteria capable of secreting single chain antibodies and antibody fragments directed to Toll-Like Receptors. Compositions comprising the transgenic bacteria and method of preventing and treating infectious, inflammatory and proliferative conditions are also provided.

### FIELD OF THE INVENTION

The present invention relates to antibody-expressing probiotic bacteria, to pharmaceutical compositions comprising them and to their use in manipulating pathogenic infections or treating inflammatory conditions and gastrointestinal cancers.

### BACKGROUND OF THE INVENTION

Toll-like receptors (TLRs) govern protective innate immune responses to a vast array of infectious agents. However, the inflammatory response orchestrated by TLRs can trigger life-threatening conditions, such as septic shock, in the setting of acute infection, and can damage host tissue in the setting of chronic infection. In periodontal disease for example, a bacterially-induced chronic inflammatory process, it was demonstrated that Toll-like receptor 2 (TLR2) plays a significant role in promoting the disease (Burns E. et al., J. Immunol., 2006, 177(12):8296-8300; Burns E. et al., J. Immunol., 2010, 184(3):1455-1462). Another example is sepsis, where it was demonstrated that TLR blockade prevents an over-abundant host response and provides protection from septic shock upon acute infection (Meng G. et al., J. Clin. Invest., 2004, 113(10):1473-1481; Daubeuf B. et al., J. Immunol., 2007, 179(9):6107-6114).

Periodontal disease is associated with the prolonged presence of bacteria and their products, alongside an unrelenting host response indicated by a dense leukocyte infiltrate and abundant pro-inflammatory mediators. The host response to infection culminates in osteoclast activation and alveolar bone resorption, the hallmark of periodontal disease. Chronic inflammation secondary to periodontal disease is linked to multiple systemic inflammatory conditions, and impacts on systemic diseases such as diabetes and cardiovascular disease. Specific gram-negative anaerobic bacteria such as P. gingivalis are major inducers of periodontal disease. Of note, P. gingivalis persists and replicates in the presence of a robust host inflammatory response. It was previously demonstrated that TLR2^{-/-} mice produce less proinflammatory cytokines in response to infection with P. gingivalis, and are resistant to alveolar bone loss following oral infection with P. gingivalis (Burns E. et al., 2006, ibid). Thus, although P. gingivalis produces multiple molecular patterns that potentially activate different pattern recognition receptors (PRRs), TLR2 dominates P. gingivalis recognition and inflammatory response. However, TLR2 is not required to overcome infection, but rather impairs host defense since TLR2 deficient mice rapidly clear P. gingivalis infection.

Up till now, the treatment of periodontal disease began with the removal of subgingival calculus (tartar) and biofilm deposits. A dental hygienist procedure called scaling and root planning is the common first step in addressing periodontal problems, which seeks to remove calculus by mechanically scraping it from tooth surfaces. Dental calculus, commonly known as tartar, consists almost entirely of calcium phosphate salt, the ionic derivative of calcium phosphate (the primary composition of teeth and bone). Dental calculus deposits harbor harmful bacteria. Clinically, calculus stuck to teeth appears to be hardened to the point requiring mechanical scraping for removal. Further, as the bacteria responsible for most periodontal disease are anaerobic, oxygenation is used to reduce bacteria populations. Thorough brushing with dilute hydrogen peroxide, with emphasis on the gum line, and flossing, helps prevent the formation of harmful biofilm, gingivitis, and tartar. Therapeutic mechanical delivery of hydrogen peroxide to subgingival pockets can be provided by a water pick. Enzymatic agents found in commercial preparations can loosen, dissolve, and prevent biofilm formation. Beneficial agents include lysozyme, lactoperoxidase, glucose oxidase, mutanase, and dextranase. Another method for treatment of periodontal disease involves the use of an orally administered antibiotic, Periostat (Doxycycline). However, Periostat does not kill the bacteria, as it only inhibits the body's host response to destroy the tissue.

PCT publication WO 96/040947 relates to recombinant bacterial system with environmentally-limited viability. PCT publication WO 2002/090551 relates to a recombinant Lactococcus that can only survive in a medium, where well-defined medium compounds are present. PCT publication WO 2005/028509 relates to antibodies that specifically bind TLR2-mediated immune cell activation. PCT publication WO 2013/149111 relates to antibodies that specifically bind Toll-like receptor 4 (TLR4). PCT publication WO2004/046346 discloses recombinant Lactobacillus strains with limited growth and viability and dependence on defined medium compounds for survival, for treatment of inflammatory bowel diseases.

There is unmet need for anti-Toll receptors 2 and 4 that could be used to treat inflammatory and cancerous conditions in the gastrointestinal system.

### SUMMARY OF THE INVENTION

The present invention provides transgenic bacteria, derived from non-pathogenic bacterial strains that secretes therapeutically-effective amounts of single-chain antibodies directed to Toll-like receptors. The present invention also provides compositions comprising the transgenic bacteria, and their use for treatment of inflammatory and proliferative conditions. The scAb-producing bacteria of the present invention are easily produced, readily isolated and administered and can be used as one-time treatment, to maintain long-term activity and/or to ensure biologic containment.

To produce antibody-secreting bacteria, probiotic bacteria was engineered by fusing polynucleotide sequences encoding variable regions (antigen binding portions) of blocking anti-TLR monoclonal antibodies (mAbs), to form single chain antibody (scAb) constructs. These constructs were overexpressed in commensal Lactobacillus bacteria to secrete the inhibitory single chain antibodies.

The present invention provides, in one aspect, a transgenic bacterium capable of expressing and secreting a single chain antibody (scAb) or an antigen-binding fragment thereof that specifically recognizes a Toll-like receptor (TLR).

In certain embodiments, the scAb or the antigen-binding fragment thereof is expressed in the transgenic bacterium from an exogenous expression cassette comprising a transcribable polynucleotide encoding the amino acid sequence of the scAb polypeptide or the amino acid sequence of the antigen-binding fragment thereof. In certain embodiments, the exogenous expression cassette comprising a transcribable polynucleotide encoding the amino acid sequence of the scAb or of the antigen-binding fragment thereof is operably linked to an expression control sequence. In certain embodiments, the expression control sequence comprises a constitutive promoter. In certain embodiments, the expression control sequence comprises an inducible promoter. In certain embodiments, the exogenous expression cassette is carried by a plasmid. In certain embodiments, the exogenous expression cassette is integrated to the bacterial genome.

In certain embodiments, the scAb or the antigen-binding fragment thereof is directed to an epitope from the extracellular domain of the TLR. In certain embodiments, the TLR is a mammalian TLR. In certain embodiments, the TLR is a human TLR. In certain embodiments, the scAb or fragment thereof is directed to a TLR expressed by a mammalian cell selected from the group consisting of a macrophage, a neutrophil, a dendritic cell, a mast cell, a T cell, a fibroblast and an epithelial cell. In certain embodiments, the scAb or fragment thereof is directed to a TLR expressed by a mammalian a macrophage. In certain embodiments, the scab or fragment thereof is directed to a human TLR selected from the group consisting of TLR2 and TLR4.

In certain embodiments, the scAb or fragment thereof is directed to TLR2. In certain embodiments, the scAb directed to TLR2 comprises the six complementarity determining regions (CDRs) contained in SEQ ID NO: 1. In certain embodiments, the scAb directed to TLR2 comprises the three heavy-chain CDR sequences set forth in SEQ ID NOs: 3-5. According to other embodiments, the scAb comprises, the three light-chain CDR sequences set forth as SEQ ID NOs: 6-8. In certain embodiments, the scAb directed to TLR2 comprises the six CDR sequences set forth in SEQ ID NOs: 3-8. In certain embodiments, the scAb directed to TLR2 comprises the amino-acid sequence set forth in SEQ ID NO: 1. In some specific embodiments, the scAb directed to TLR2 consists of the amino-acid sequence set forth in SEQ ID NO: 1.

In certain embodiments, the scAb or fragment thereof is directed to TLR4. In certain embodiments, the scAb directed to TLR4 comprises six CDRs contained in SEQ ID NO: 2. In certain embodiments, the scAb directed to TLR2 or the fragment thereof, comprises the three heavy-chain CDR sequences set forth in SEQ ID NOs: 9-11. According to other embodiments, the scAb directed to TLR2 or the fragment thereof, comprises the three light-chain CDR sequences set forth in SEQ ID NOs: 12-14. In certain embodiments, the scAb directed to TLR2 or the fragment thereof comprises the six CDR sequences set forth in SEQ ID NOs: 9-14. In certain embodiments, the scAb directed to TLR4 comprises or consists of the amino-acid sequence set forth in SEQ ID NO: 2.

Determination of CDR sequences can be made according to any method known in the art, including but not limited to the methods known as KABAT, Chothia and IMGT. A selected set of CDRs may include sequences identified by more than one method, namely, some CDR sequences may be determined using KABAT and some using IMGT, for example.

In certain embodiments, the antigen-binding fragment comprises the VH region of the antibody and the VL region of the antibody. According to some specific embodiments the antigen-binding fragment consists of the hypervariable region of the antibody.

In certain embodiments, the scAb molecule is composed of a heavy-chain and light-chain variable regions connected directly or through a spacer or a linker. In certain embodiments, the linker consists of 3-30 amino acids. In other embodiments, the linker consists of 10-20 amino acids. In certain embodiments the linker comprises at least two Glycine (Gly, G) residues and at least two Serine (Ser, S) residues. In some specific embodiments, the linker comprises the sequence GGGGSGGGGSGGGGS (SEQ ID NO: 15). In yet other specific embodiments, the linker consists of the sequence GGGGSGGGGSGGGGS (SEQ ID NO: 15).

In certain embodiments, the transgenic bacterium constantly expresses and secretes the scAb or the antigen-binding fragment thereof. In other embodiments, the scAb or the antigen-binding fragment thereof is expressed on the surface of the bacteria.

In certain embodiments, the transgenic bacterium is capable of reproduction in the body of a mammalian subject, on a mucosal surface of a mammalian subject or on a skin of a mammalian subject. According to some embodiments, the mucosal surface of a mammalian subject is selected from the group consisting of: oral mucosa, nasal mucosa, gastrointestinal mucosa, vaginal mucosa and urinary bladder mucosa. In certain embodiments, the transgenic bacterium is auxotroph and is incapable of reproduction on a mucosal surface or in the body of a subject because of a biocontainment strategy applied. In certain embodiments, the transgenic bacterium is probiotic, commensal, mutualistic or non-pathogenic in mammals. In certain embodiments, the transgenic bacterium is commensal in mammals, including but not limited to humans. In certain embodiments, the transgenic bacterium is of the order Lactobacillales. In certain embodiments, the transgenic bacterium is of the family Lactobacillaceae. In certain embodiments, the transgenic bacterium is of the genus Lactobacillus.

In certain embodiments, a biocontainment strategy is used to prevent the continued reproduction, divisional or proliferation of the transgenic bacteria in the mammalian host. Biocontainment can be achieved in several ways, all which are included in the scope of the present invention to provide biocontainment bacteria. Examples for method for inductions of biocontainment strategies according to some embodiments of the present inventions, include but are not limited to introducing a suicide gene to the bacteria that is kept in an “off” state by a factor supplied to the bacteria when they are grown in culture, but that is not present in a healthy mammalian body or on the mucosal surface of the mammalian subject. According to some embodiments, the transgenic bacteria lacks an active essential gene product, and depends on the presence of said gene product in the growing medium or in the treated mammalian body. Such bacteria are known as auxotrophs, and methods to create auxotrophs are known in the literature and to those skilled in the art. According to some specific embodiments, the essential gene is inactivated by deletion or replacement of a polynucleotide sequence. According to some specific embodiments, the bacteria lack an active thymidylate synthase gene and its growth depends on the presence of thymidine and/or thymine. According to other specific embodiments, the bacteria are gram positive bacteria that require D-alanine for growth, and the transgenic bacteria lack an active alanine racemase gene, rendering their growth dependent on the presence of D-alanine.

Polynucleotide sequences comprising sequences that encode scAb and binding fragments thereof that specifically recognize mammalian TLR, are also within the scope of the present invention. According to some embodiments, a polynucleotide sequence is provided comprising a sequence that encodes a scAb or a fragment thereof, and a bacterial promoter sequence. The sequence that encodes a scAb or fragment thereof can be introduced to the bacterial genome with a promoter sequence or can be expressed under the control of an endogenous promoter. According to some embodiments, the polynucleotide sequence encodes a polypeptide sequence set forth in SEQ ID NO: 1. According to some embodiments, the polynucleotide sequence encodes a polypeptide sequence set forth in SEQ ID NO: 2.

Another aspect of the invention relates to a transformed strain of bacteria, comprising a gene or expression unit encoding a scAb specific to TLR. In certain embodiments, the transgenic bacterium is of the order Lactobacillales. In certain embodiments, the transgenic bacterium is of the family Lactobacillaceae. According to some embodiments, the bacteria is of the genus Lactobacillus. According to some embodiments, the TLR is a mammalian TLR4 or TLR2. According to some embodiments, the transformed bacterial strain comprises a gene or expression unit encoding a scAb comprising a set of six CDR sequences set forth in SEQ ID Nos. 3 to 8. According to some embodiments, the transformed bacterial strain comprises a gene or expression unit encoding a sequence set forth as SEQ ID NO: 1. According to some embodiments, the transformed bacterial strain comprises a gene or expression unit encoding a scAb comprising a set of six CDR sequences set forth in SEQ ID Nos. 9 to 14. According to some embodiments, the transformed bacterial strain comprises a gene or expression unit encoding a sequence set forth as SEQ ID NO: 2.

The present invention further provides, according to another aspect, a composition comprising transgenic bacteria, the bacteria comprising at least one polynucleotide sequence encoding a scAb directed to a TLR or a binding fragment thereof, wherein the bacteria is capable of secreting the scAb or the scAb fragment.

In certain embodiments, the composition comprises at least two different kinds of bacteria, wherein each kind of bacterium expresses and is capable of secreting a different scAb or an antigen-binding fragment thereof. According to some embodiments, the composition comprises bacteria expressing at least two scAb molecules or binding fragments thereof, each is directed to a different TLR. According to some specific embodiments, the composition comprises bacteria expressing a scAb to TLR2 and a scAb to TLR4 or binding fragments thereof.

In certain embodiments, the composition further comprises an additional active agent. According to some embodiments, the active agent is selected from the group consisting of: an anti-pathogenic agent, an anti-inflammatory agent and an anti-cancer agent.

In certain embodiments, the composition is formulated for mucosal delivery. In certain embodiments, the composition is formulated for oral delivery. In yet other embodiments, the composition is formulated for topical delivery. In some embodiments, the composition is formulated, for example by encapsulation, for a controlled or sustained release. In some embodiments, the bacteria in encapsulated, using methods known in the art, to be released from the capsules in specific areas of the gastrointestinal tract (GI). In certain embodiments, the composition is formulated as a nutraceutical product. In certain embodiments, the composition is formulated for rectal delivery. According to some embodiments, the composition is formulated for vaginal delivery. In certain embodiments, the composition is formulated as a suppository or as enema. In certain embodiments, the composition is formulated as a solid, a gel, a paste or an ointment. According to some specific embodiments, the composition is formulated for intravesicular administration. In certain embodiments, the composition is formulated as a tooth paste or as an oral rinse. In yet other embodiments, the composition is formulated as a liquid, semi-liquid or a suspension.

In certain embodiments, the composition comprises 10⁶ to 10¹² colony-forming-units (c.f.u.) of the transgenic bacterium.

In certain embodiments, a composition comprising bacteria expressing at least one scAb is provided, for use in inhibiting TLR signaling in mammalian cells. According to some embodiments, the cells are leukocytes or epithelial cells. In certain embodiments, the composition is for use in increasing an immune response in a subject towards a pathogen. In certain embodiments, the pathogen comprises or secretes a ligand which activates a TLR. In certain embodiments, the pathogen is a bacterial pathogen. In certain embodiments, the bacterial pathogen is of the genus Porphyromonas. In certain embodiments, the bacterial pathogen is of the species Porphyromonas gingivalis. In certain embodiments, the transgenic bacterium is capable of expressing and secreting a scAb or an antigen-binding fragment thereof directed to TLR2. In certain embodiments, the transgenic bacterium is of the genus Lactobacillus.

In certain embodiments, a composition according to the present invention is for use in preventing or ameliorating inflammation in a subject. In certain embodiments, the inflammation is associated with a gastrointestinal disease or disorder. In certain embodiments, the inflammation is associated with an oral or periodontal disease or disorder. In certain embodiments, the inflammation is associated with radiation-induced proctitis. In certain embodiments, the inflammation is associated with inflammatory bowel disease (IBD). In certain embodiments, the inflammation is associated with Crohn's disease. In certain embodiments, the inflammation is associated with ulcerative colitis. In yet other embodiments, the inflammation is associated with cancer of the gastrointestinal system. In other embodiments, the inflammation is associated with a genitourinary disease or disorder, including but not limited to urinary cancer, bladder cancer, prostate cancer, genital infection and urinary tract infection (UTI).

In certain embodiments, the inflammation is associated with an endogenous ligand, including but not limited to a ligand generated by necrotic cell death. In certain embodiments, the inflammation is associated with an exogenous ligand including but not limited to lipopolysaccharide (LPS), and lipopeptide products of bacterial cells.

In certain embodiments, a composition according to the present invention is for use in reducing secretion of an inflammatory cytokine by a mammalian cell. According to some embodiments, the inflammatory cytokine is selected from the group consisting of: interferon gamma (IFN-gamma, IFNγ), interleukin 1 beta (IL-1 beta, IL-1β), tumor necrosis factor alpha (TNF alpha, TNFα), and interleukin 6 (IL-6). According to some specific embodiments, the inflammatory cytokine is TNFα. In certain embodiments, a composition according to the present invention is for use in increasing phagocytosis of a pathogen by a mammalian cell. In certain embodiments, the mammalian cell is a cell selected from the group consisting of a macrophage, a neutrophil, a dendritic cell, a mast cell, a T cell, and a fibroblast. In certain embodiments, the cell is a macrophage. In certain embodiments, the composition is for inhibiting the response of a mammalian cell to stimulation by a ligand which activates a TLR.

According to some embodiments, the composition is for use in preventing or treating an inflammatory or proliferative disease of the gastrointestinal (GI) system. In certain embodiments, the composition is for preventing or treating gastrointestinal cancer in a subject. In some embodiments, the GI cancer is selected from colon cancer, gastric cancer, esophageal cancer, and adenocarcinoma According to some embodiments, the gastrointestinal cancer is colon cancer.

According to some embodiments, the composition is for use in preventing or treating IBD e.g. Crohn's disease and colitis. According to some embodiments, the treatment results in improvement of at least one symptom of gastrointestinal inflammation and IBD. According to some embodiments, the treatment result in improvement of subject's weight, e.g. decreased weight loss. According to some embodiments, the treatment result in decrease in rectal bleeding.

According to other embodiments, the composition is for use in preventing or treating a genitourinary infection or inflammation.

In certain embodiments, use of a composition according to the present invention for preventing or treating a disease comprises applying the composition to a mucosal surface of a subject, in the form of an oral rinse, a dentifrice, a troche, a capsule, an enema or a suppository. In certain embodiments, the composition is formulated for oral or topical delivery.

A composition of the present invention may be delivered to the treated tissue or cells by any method known in the art. According to some embodiments, the composition comprises encapsulated bacteria to allow sustained release and/or to improve the delivery, e.g. to the gastric surface or to specific parts of the GI system, for example to the intestine.

The present invention further provides, in another aspect, a method of inhibiting TLR signaling in a mammalian cell in a subject in need thereof, comprising the step of administering any one of the compositions described above to the subject, thereby inhibiting TLR signaling. According to some embodiments, the mammalian cell is a leukocyte or an epithelial cell.

The present invention further provides, in another aspect, a method of preventing or treating a disease or disorder of the gastrointestinal system or of the genitourinary system, comprising administering to a subject in need thereof a composition comprising transgenic bacteria capable of secreting a scAb against a mammalian TLR, thereby treating the disease or disorder.

According to some embodiments, the disease or disorder of the gastrointestinal system is selected from the group consisting of: a pathogenic infection, an inflammatory reaction and a proliferative disease.

According to some embodiments, the disease or disorder of the genitourinary system is selected from the group consisting of: a pathogenic infection, an inflammatory reaction and a proliferative disease.

The invention thus provides, according to an aspect, a method of treating an infection with a specific micro-organism, or a condition that is associated with an unhealthy microbiota. According to some embodiments, the condition is a disease or disorder of the gastrointestinal system in which the gut microbiome is different from that of healthy subjects. According to other embodiments, the condition is a disease or disorder of the genitourinary system in which a vaginal or urinary microbiome is different from that of healthy subjects. According to some embodiments, the method comprises the step of administering a composition comprising transgenic bacteria expressing at least one scAb directed against a mammalian TLR to the subject, thereby treating the infection or restoring the microbiota to a health-associated microbiota. According to some embodiments, the condition of the gastrointestinal system is selected from the group consisting of: periodontal disease, IBD and cancer. According to some embodiments, the cancer is colon cancer. According to some embodiments, the condition of the genitourinary system is selected from the group consisting of infection and cancer. According to some embodiments, the genitourinary cancer is selected from bladder cancer and prostate cancer.

The present invention further provides, in another aspect, a method of treating an inflammation in a subject in need thereof, comprising the step of administering a composition comprising transgenic bacteria expressing at least one scAb directed against a mammalian TLR to the subject, thereby treating the inflammation. In certain embodiments, the inflammation is associated with a gastrointestinal disease or disorder. In certain embodiments, the inflammation is associated with an oral or periodontal disease or disorder. In certain embodiments, the inflammation is associated with radiation-induced proctitis. In certain embodiments, the inflammation is associated with inflammatory bowel disease (IBD). In certain embodiments, the inflammation is associated with Crohn's disease. In certain embodiments, the inflammation is associated with ulcerative colitis. In yet other embodiments, the inflammation is associated with cancer of the gastrointestinal system. In yet other embodiments the inflammation is associated with the genitourinary system.

According to some embodiments a method of preventing inflammation in the oral cavity is provided. According to some embodiments, a method of preventing or treating an oral or periodontal inflammation is provided comprising administering a composition comprising transgenic bacteria expressing at least one scAb directed against a mammalian TLR to the subject. According to some embodiments, the periodontal inflammation is associated with P. gingivalis. According to other embodiments the periodontal inflammation is associated with bacteria other than P. gingivalis. According to some embodiments, the inflammation develops in the area of a dental implant and is referred to as peri-implant disease that includes peri-implant mucositis (involving the soft tissues around the implant), and peri-implantitis (involving both the soft tissue and bone around the implant). According to some embodiments, the treatment results in reduced alveolar bone resorption and/or increased bone volume.

The present invention further provides, in another aspect, a method of preventing or treating a gastrointestinal cancer in a subject in need of such treatment, the method comprising the step of administering a composition comprising transgenic bacteria expressing at least one scAb directed against a mammalian TLR to the subject, thereby treating the cancer. According to some embodiments, the cancer is colon cancer.

According to some embodiments, a method of preventing or treating IBD is provided comprising the step of administering a composition comprising transgenic bacteria expressing at least one scAb directed against a mammalian TLR to the subject. According to some embodiments, the IBD is selected from Crohn's disease and colitis. According to some embodiments, the treatment results in improvement of weight loss, rectal bleeding and/or other clinical signs and symptoms of the disease.

In certain embodiments, a composition according to the present invention is for use in preventing or ameliorating inflammation in a subject. According to some embodiments, the preventing or treating method comprises the step of administering a composition according to the invention via an administration route selected from the group consisting of: mucosal, oral, topical, rectal (including but not limited to administration as suppository or as enema), vaginal and intravesicular. According to some embodiments, the method of preventing or treating comprises the step of applying the composition to a mucosal surface of a subject. According to some embodiments, the mucosa is selected from the group consisting of: oral mucosa, nasal mucosa, gastrointestinal mucosa, vaginal mucosa and urinary bladder mucosa. According to some embodiments, an encapsulated composition is administered.

According to some embodiments, the treatment is with transgenic bacteria that can replicate in the human host following treatment. According to other embodiments, the treatment is with transgenic bacteria designed to be dependent on a compound not found naturally in healthy humans in sufficient amounts to support continued replication of the bacteria. According to some embodiments, the transgenic bacteria lacks an active essential gene product, and dependents on the presence of said gene product in the growing medium or in the treated mammalian body. According to some specific embodiments, the essential gene was inactivated by deletion or replacement of a polynucleotide sequence. According to some specific embodiments, the bacteria lack an active thymidylate synthase gene and its growth depends on the presence of thymidine and/or thymine. According to other specific embodiments, the bacteria are gram positive bacteria that require D-alanine for growth, and the transgenic bacteria lack an active alanine racemase gene, rendering their growth dependent on the presence of D-alanine.

Any treatment regimen known in the art against inflammation, infection or cancer may be used in combination with the treatment methods of the present invention, including but not limited to administration of active agents, surgery and radiation.

According to some embodiments, the method of treating cancer comprises administering or performing at least one additional anti-cancer therapy. According to certain embodiments, the additional anticancer therapy is surgery, chemotherapy, radiotherapy, or immunotherapy.

According to some embodiments, the method of treating cancer comprises administration of a composition comprising anti TLR scAb-secreting bacteria and an additional anti-cancer agent. According to some embodiments, the additional anti-cancer agent is selected from the group consisting of: immune-modulator, activated lymphocyte cell, kinase inhibitor and chemotherapeutic agent.

According to other embodiments, the additional immune-modulator is an antibody, antibody fragment or antibody conjugate that binds to an antigen other than TLR. The additional anti-cancer agent may be administered using a different mode, for example enterally or parenterally.

According to some specific embodiments, the cancer is bladder cancer and the administration is intravesicular. According to some embodiments, the cancer is a gastrointestinal cancer, e.g. colon cancer.

The bacterial compositions of the present invention may be delivered to a subject in need thereof by any delivery or administration route suitable for delivery of bacteria, e.g. probiotic bacteria. According to some embodiments, the composition is delivered to a mucosal tissue of the treated body. According to some embodiments, the compositions are delivered orally or topically for example as tooth paste, oral rinse, dentifrice, troche, capsule, enema or suppository, or topically applied to the skin. According to some embodiments, the bacterial composition is administered as a nutraceutical product. According to some embodiments, the nutraceutical product comprising bacteria of the present invention, comprises at least one additional active ingredient. In certain embodiments, the composition is administered rectally or vaginally. In other embodiments, the administration is intravesicular. In certain embodiments, the composition is administered by a suppository or enema. In certain embodiments, the composition is administered as a solid, gel, a paste or an ointment. In other embodiments, the composition is administered as a liquid, semi liquid or a suspension.

According to some specific embodiments, a method of treating a disease of the gastrointestinal system is provided the method comprising rectal administration of a composition comprising anti-TLR scAb-secreting bacteria. According to some embodiments the gastrointestinal disease is IBD or colon cancer. According to certain embodiments, the rectal administration is via a suppository or enema.

According to some specific embodiments, a method of treating a disease of the genitourinary system is provided the method comprising administration of a composition comprising anti-TLR scAb-secreting bacteria. According to some embodiments the genitourinary disease is cancer. According to certain embodiments, the cancer is bladder cancer and administration is intravesicular. According to some embodiments the administration is vaginal. According to other specific embodiments, a method of treating an inflammation in the gastrointestinal system is provided the method comprising oral administration of a composition comprising anti-TLR scAb-secreting bacteria. According to some embodiments the gastrointestinal inflammation is oral and the composition is administered orally, e.g. as an oral rinse or as a tooth paste. According to certain embodiments, administration is during a tooth or dental implant treatment, operation or surgery.

The present invention also provides, according to another aspect, a kit comprising transgenic bacteria expressing scAb that specifically recognize TLR. According to some embodiments, the kit comprises a container with the transgenic bacteria and a separate container comprising an agent capable of activating the biocontainment strategy. According to some embodiments, the kit further comprises instructions of using the individual containers. According to some embodiments, the kit comprises transgenic bacteria comprising a polynucleotide sequence encoding a polypeptide of SEQ ID NO: 1 or SEQ ID NO: 2.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a bar graph illustrating the effects of TLR inhibition on macrophage response to P. gingivalis. FIG. 1A: Bone marrow macrophages (BMM) from wild-type mice were challenged with live P. gingivalis in the presence of inhibitory antibodies to TLR2 (clone T2.5) or TLR4 (clone 1A6) or isotype control. FIG. 1B: BMM were challenged with FITC-labeled P. gingivalis and phagocytosis was measured after quenching extracellular florescence with Trypan Blue. FIG. 1C: BMM were challenged with P. gingivalis in the absence of antibodies (NT) or in the presence of anti-TLR2, anti-TLR4, or isotype control. BG—Background. Pg—P. gingivalis. NT—Not treated.

FIG. 2 is an illustration of the DNA plasmid for overexpression of single chain antibody (scAb) constructs in Lactobacillus.

FIG. 3 is a picture of a PCR gel showing the amplicons from Lactobacillus transformants of scAb against TLR2 (lanes 1-4), and TLR4 (lanes 7-8). The PCR product size matches the size amplified from the respective plasmid DNA shown in lanes 10 (TLR2 scAb plasmid) and lane 11 (TLR4 scAb plasmid). Plasmid DNA without a scAb insert (lane 12) does not produce a band, and there are no bands present in the colony PCR of Lactobacillus transformed with this DNA (lanes 5-6). Lane 9 represents a negative control (no template DNA).

FIG. 4 is a picture of an anti-His Western blot (WB) showing bands at the predicted molecular weight. Supernatants of different transformants were TCA precipitated and precipitates were tested by WB: (1) TLR2 scAb transformants, (2) an empty vector (EV) transformant, and (3) TLR4 scAb transformants.

FIG. 5 is a bar graph illustrating the levels of TNFα secreted from (FIG. 5A) RAW 264.7 or (FIG. 5B) THP-1 cells. P value <0.03 (*, **). BG—Background. EV—Empty plasmid vector.

FIG. 6A is a scatter graph illustrating the c.f.u. of P. gingivalis as determined on blood agar plates from mice which received one of three different types of transformed lactobacilli prior to infection with the P. gingivalis bacteria. FIG. 6B is a bar graph illustrating the TNF levels in the exudates determined by ELISA. **P<0.01 compared to EV at the same time point.

FIGS. 7A-7D shows a series of immunofluorescence images of gingival tissue prepared by whole-mount to detect binding of scaTLR2 to oral tissue. Mice were lavaged once with 10⁹ bacteria/mouse of three different types of transformed lactobacilli: Lactobacillus empty vector (EV, FIG. 7A), Lactobacillus single chain antibody against human TLR4 (scahTLR4, FIG. 7B), or Lactobacillus single chain antibody against human TLR2 (scaTLR2, FIGS. 7C-7D). Mice were sacrificed after 24 h and maxillary gingival tissues were processed for whole mounting and stained with anti-His (white) FIGS. 7A-7C, 4X. FIG. 7D, 20X.

FIG. 8 shows Tnf, Il1b, and Il17 gene expression levels measured by qPCR on the gingiva of mice from different groups. Mice were lavaged in the oral cavity three times with CMC (carboxy-methyl cellulose, carrier control) vs. P. gingivalis in CMC every other day. Groups of mice challenged with P. gingivalis were administered Lactobacillus secreting single chain antibody against TLR2 (LactoTLR2) vs. Lactobacillus secreting single chain antibody against human TLR4 (LactoTLR4) on the intermittent days between P. gingivalis challenge. qPCR was performed on gingival tissue 72 hours after the last lavage (n=6 per group).

FIG. 9A shows Rankl/Opg gene expression levels measured by qPCR on the gingiva of mice from different groups. Mice were lavaged in the oral cavity three times with CMC (carboxy-methyl cellulose, carrier control) vs. P. gingivalis in CMC every other day. Groups of mice challenged with P. gingivalis were administered Lactobacillus empty vector (EV) vs. Lactobacillus secreting single chain antibody against human TLR2 (scaTLR2) in the oral cavity on the intermittent days between P. gingivalis challenge. qPCR was performed on gingival tissue 6 weeks after the last lavage (n=12 per group). FIG. 9B shows the results of a μCT analysis to measure residual bone volume around the second maxillary molar on the same mice 6 weeks after the last challenge (n=12 per group).

FIG. 10A shows the average distance from the cemento-enamel junction (CEJ) to the alveolar bone crest (ABC) on the buccal surface of the maxilla. The CEJ-ABC distance was measured at six sites and an average distance was determined. Mice were divided into four groups of five mice each. 5-0 silk ligatures were tied around the maxillary left second molar in three of the groups and in one group no ligature was tied. Following ligature placement, mice were administered CMC alone (groups “CMC” and “Ligature only”) vs. Lactobacillus secreting scaTLR2 in CMC (group “Ligature and aTLR2), vs. Lactobacillus transformed with the empty vector plasmid (“Ligature and EV” group). The treatments were performed once daily for four days and mice were sacrificed on day 7. The difference in CEJ-ABC average distance was compared between each group to the CMC group that did not have a ligature placed. ***P<0.005, *P<0.05, ns, not significant. FIG. 10B shows Rankl/Opg gene expression levels measured by qPCR on the gingiva of the same mice from the different groups. * P<0.05, ns—not significant.

FIG. 11A shows the percent weight loss of five groups of mice (n=6 per group) subjected to the DSS-colitis model of acute inflammatory bowel disease. 2.5% Dextran sodium sulfate (DSS) was administered in the drinking water to four groups of mice from day 0 to day 5. One group of mice served as a control group and received regular water. On days 2, 3, and 4, three groups of mice were administered different Lactobacilli by gastric gavage. One group of mice received Lactobacilli secreting single chain anti-TLR2 (Lacto scaTLR2), one group received Lactobacilli secreting single chain anti-murine TLR4 (Lacto scaTLR4), and one group received Lactobacilli transformed with the empty vector plasmid (Lacto EV). Mice were sacrificed on day 5. *P<0.05, comparison of weight of DSS only group to DSS+Lacto scaTLR2 group. FIG. 11B shows the average length of the colon of the same mice on day 5. All statistical comparisons are to the DSS only group. **P<0.01, ***P<0.005.

### DETAILED DESCRIPTION OF THE INVENTION

The present invention provides bacteria capable of secretion of therapeutically-effective amounts of single-chain antibodies (scAb) against Toll-like receptors (TLRs). The provision of such bacteria is beneficial in several fields, including prevention, treatment, and prevention of recurrence of any adverse conditions, diseases or disorders which are associated with, involving, induced by or dependent of TLR signaling. Specifically, the provision of antibody-secreting bacteria according to the present invention allows, for the first time, the prevention and treatment of a variety of adverse TLR-related conditions by one-time or continuous administration of the bacteria to the site to be treated or protected and short-term, containment, long-term and/or induced secretion of the active scAb directly or close to the diseased tissue. According to the principles of the present invention, it is noted that the amount of therapeutic anti-TLR antibodies at the site of administration, as well as the boundaries of the site to be treated and schedule of release of active scAb, can be predetermined and/or manipulated to achieve an optimal therapeutic outcome.

The advantages of employing scAb-secreting bacteria, e.g. to prevent or ameliorate inflammation in the host, instead or in addition to purified full-size monoclonal antibodies (mAbs), are several. First, the need for elaborate, expensive and time-consuming Ab-purification and concentration techniques is eliminated, as antibody-secreting bacteria according to the present invention are readily isolated. Secondly, the need to predetermine the exact therapeutic dose of the administered antibody is eliminated, since antibody-secreting bacteria according to the present invention allow the in-vivo manipulation of the level of mAb delivered to the subject, for example using an auxotroph that will not replicate in the mammalian subject. Using an auxotroph enables delivery of the scAb over a short term. Furthermore, the half-life of a scAb is much shorter than the half-life of full-size mAb, as is known in the literature. Thirdly, the need for repeated administration of mAbs, in case the initial dose is found not to be sufficient, is also potentially eliminated, for the same reason. Another advantage is the ability to substantially spatially-focus the treatment—as mAb are usually administered systemically, antibody-secreting bacteria according to some embodiments of the present invention are administered to mucosal surfaces, on which they are stationary. Yet another advantage is the possibility of inducing and maintenance of long-term protection, if needed. While administered mAb have a limited in-vivo half-life, antibody-secreting bacteria according to some embodiments of the present invention can be replication-competent and form part of the natural microbiota of the host. Thus, they can remain within and protect the host against future challenges e.g. prevent recurrence.

In certain embodiments, the viability of the transgenic bacterium in a mammal host, including in human, is dependent on a compound not found naturally in healthy humans and the bacteria are therefore contained and can be used to deliver the scAb according to a predetermined dosing regimen, similar to classical drug dosing.

Various systems can be used, according to some embodiments of the present invention, to ensure biologic containment of engineered bacteria and adapt it for a short-term use if necessary, including but not limited to the methods reviewed in Torres et al Essays in biochemistry 2016, 60, 393-410). One method that may be used according to some embodiments of the present invention is induced auxotrophy. In this approach the ability of the engineered bacteria to synthesize a vital compound is removed, such that the compound must be acquired from the organism's growth media or the environment. One prominent example of this approach disclosed in WO2014/046346, is to inactivate (preferably by deletion or replacement) the thymidylate synthase gene. These bacteria are dependent on the presence of thymidine and/or thymine for growth and cannot persist in the human body. Such methods to engineer and use a thymidine auxotroph may be utilized for the anti-TLR scAb-secreting bacteria of the present invention. According to some specific embodiments, the bacteria lack and active thymidylate synthase gene and its growth depends on the presence of thymidine and/or thymine.

The present invention provides, in one aspect, a transgenic bacterium capable of expressing and secreting an exogenous single chain antibody (scAb) or an antigen-binding fragment thereof directed to a Toll-like receptor (TLR).

The term “single chain antibody” or “scAb” as used herein refers to a single polypeptide chain containing one or more TLR-binding domains that bind an epitope of a TLR.

The phrase “antigen-binding fragment” as used herein refers to a TLR-binding domain that binds an epitope of a TLR.

The term “Toll-like receptor” or “TLR” as used herein generally refers to a membrane-spanning, non-catalytic receptor protein, which recognizes at least one structurally-conserved molecule derived from microbes. The human TLRs include TLR1-TLR12. For example, human TLR2 is identified by the UniProt number 060603, and human TLR4 is identified by the UniProt number 000206.

In certain embodiments, the transgenic bacterium constantly expresses and secretes the scAb or the antigen-binding fragment thereof. The phrase “constantly expresses and secretes” as used herein refers to expression and secretion without dependency on external stimulus.

In certain embodiments, the transgenic bacterium expresses and secretes the scAb or the antigen-binding fragment thereof in response to an external stimulus, using an inducible promoter. The term “external stimulus” as used herein refers to a signal external to the bacteria, such as an environmental, biological or chemical signal to which expression and/or secretion are functionally linked.

According to the principle of the present invention, non-pathogenic bacteria are genetically manipulated to express and secrete anti-TLR scAbs. In certain embodiments, the scAb or the antigen-binding fragment thereof is expressed from an exogenous expression cassette comprising a transcribable polynucleotide encoding the exogenous scAb or the antigen-binding fragment thereof. In certain embodiments, the scAb or the antigen-binding fragment thereof is expressed from an exogenous expression cassette comprising a transcribable polynucleotide encoding the exogenous scAb or the antigen-binding fragment thereof operably linked to an expression control sequence. In certain embodiments, the expression control sequence comprises a constitutive promoter. In certain embodiments, the expression control sequence comprises an inducible promoter. In certain embodiments, the exogenous expression cassette is carried by a plasmid. In certain embodiments, the exogenous expression cassette is integrated to the bacterial genome.

In certain embodiments, the viability of the transgenic bacteria is controlled by externally-inducing expression of one or more essential genes and/or externally-inducing silencing of one or more lethal genes. In certain embodiments, temporary viability can be achieved by temporarily externally-inducing expression of one or more essential genes and/or temporarily externally-inducing silencing of one or more lethal genes. The phrase “externally-inducing” as used herein refers to exposing the transgenic bacteria to an external stimulus to which the promoter of the one or more essential genes or the promoter of the one or more lethal genes is responsive.

In certain embodiments, the transgenic bacterium is capable of reproduction on a mucosal surface of a subject. The phrase “capable of reproduction” as used herein means capable of completing at least a single step of reproduction while attached to a mucosal surface.

In certain embodiments, the transgenic bacterium is probiotic, commensal, mutualistic or non-pathogenic in humans. In certain embodiments, the transgenic bacterium is commensal in humans. In certain embodiments, the transgenic bacterium is of the order Lactobacillales. In certain embodiments, the transgenic bacterium is of the family Lactobacillaceae. In certain embodiments, the transgenic bacterium is of the genus Lactobacillus.

In certain embodiments, the scAb or the antigen-binding fragment thereof is directed to the extracellular domain of the TLR. In certain embodiments, the TLR is a human TLR. In certain embodiments, the TLR is expressed by a sentinel cell. The term “sentinel cell” as used herein broadly relates to immunological cells which embed themselves in tissues such as skin. In certain embodiments, the TLR is expressed by a cell selected from the group consisting of a macrophage, a neutrophil, a dendritic cell, a mast cell, a T cell, a fibroblast and an epithelial cell. In certain embodiments, the TLR is expressed by a macrophage. In certain embodiments, the TLR is selected from the group consisting of TLR2 and TLR4.

In certain embodiments, the TLR is TLR2. In certain embodiments, the scAb directed to TLR2 comprises three heavy-chain complementarity determining regions (HC-CDRs) of a heavy-chain variable region set forth in SEQ ID NO: 1 and three light-chain CDRs (LC-CDRs) of a light-chain variable region set forth in SEQ ID NO: 1. In certain embodiments, the scAb directed to TLR2 comprises at least one CDR sequence selected from the group consisting of the CDR sequences set forth in SEQ ID NOs: 3-8. In certain embodiments, the scAb directed to TLR2 comprises the CDR sequences set forth in SEQ ID NOs: 3-8. In certain embodiments, the scAb directed to TLR2 comprises or consists of the amino-acid sequence set forth in SEQ ID NO: 1.

In certain embodiments, the TLR is TLR4. In certain embodiments, the scAb directed to TLR4 comprises three heavy-chain complementarity determining regions (HC-CDRs) of a heavy-chain variable region set forth in SEQ ID NO: 2 and three light-chain CDRs (LC-CDRs) of a light-chain variable region set forth in SEQ ID NO: 2. In certain embodiments, the scAb directed to TLR2 comprises at least one CDR sequence selected from the group consisting of the CDR sequences set forth in SEQ ID NOs: 9-14. In certain embodiments, the scAb directed to TLR2 comprises the CDR sequences set forth in SEQ ID NOs: 9-14. In certain embodiments, the scAb directed to TLR4 comprises or consists of the amino-acid sequence set forth in SEQ ID NO: 2. In certain embodiments, the antigen-binding fragment is selected from the group consisting of a VH (variable heavy) region of the antibody, a VL (variable light) region of the antibody, a CDR (complementarity-determining region) of the VH region of the antibody, a CDR of the VL region of the antibody, and any combination thereof. Each possibility represents a separate embodiment of the invention.

Determination of CDR sequences from a given antibody or functional fragment thereof may be made according to any method known in the art, including but not limited to the methods known as KABAT, Chothia and IMGT. A selected set of CDRs may include sequences identified by more than one method, namely, some CDR sequences may be determined using KABAT and some using IMGT, for example. There are several methods known in the art for determining the CDR sequences of a given antibody molecule, but there is no standard unequivocal method. Utilization of different methods for CDR determination may result in nomination of non-identical CDR sequences from the same heavy and/or light chain variable regions.

The use of live bio-therapeutics, i.e. the treatment of an undesirable disease or condition in humans by the administration of an extrinsic population of cells, such as the bacteria and compositions of the present invention, may involve a variety of safety mechanisms to determine and control the presence (viability) and/or activity of the administered cells. The invention relates to recombinant bacteria, with environmentally limited viability and/or activity. In certain embodiments, the bacteria can only survive in a medium, where well-defined compounds are present, which are not present in the human body. The invention further relates to recombinant bacteria, with a sensitivity to one or more antibiotic agents. In certain embodiments, the viability and/or activity of the transgenic bacterium in a human host is dependent on a compound not found naturally in healthy humans. In certain embodiments, the viability and/or activity of the transgenic bacterium in a human host is limited to about 1 hour to about 1 month. In certain embodiments, the viability and/or activity of the transgenic bacterium in a human host is limited to about 1 hour to about 1 day. In certain embodiments, the viability and/or activity of the transgenic bacterium in a human host is limited to about 1 day to about 1 week. Various methods and mechanism are known to control the presence (viability) and/or activity of the bacterial cells in-vivo. The present invention further provides, in another aspect, a composition comprising bacteria, the bacteria comprising at least one transgenic bacterium as described above.

In certain embodiments, the composition comprises at least two different bacteria as described above, wherein each bacterium expresses and secretes a different scAb or an antigen-binding fragment thereof directed to a different TLR.

In certain embodiments, the composition further comprises an additional anti-pathogenic or anti-inflammatory agent. The term “anti-inflammatory agent” as used herein refers generally to any compound or combination of compounds that, upon introduction to a tissue which exhibits inflammation, tends to reduce such inflammation. In certain embodiments, the anti-inflammatory agent is an antibiotic agent. In certain embodiments, the composition further comprises scAbs or antigen-binding fragments thereof directed to a TLR outside the transgenic bacteria. In certain embodiments, the composition is substantially devoid of scAbs or antigen-binding fragments thereof outside the transgenic bacteria.

Pharmaceutical compositions according to the present invention, may comprise, in addition to the bacterium, a pharmaceutically acceptable excipient, carrier, buffer, stabilizer or other materials well known to those skilled in the art. Such materials should be non-toxic and should not interfere with the efficacy of the secreted anti-TLR scAb. The precise nature of the carrier or other material may depend on the route of administration.

In certain embodiments, the composition is formulated for mucosal delivery, namely formulated to be suitable for application to a mucosal membrane. The term “mucosal delivery” as used herein refers to the delivery to a mucosal surface, including oral, gastrointestinal, nasal, pulmonary, vaginal, rectal, urethral, sublingual or buccal delivery. In certain embodiments, the composition is formulated for oral delivery. The term “oral delivery” as used herein refers to delivery to, or via, the oral cavity. In certain embodiments, the composition is formulated as a nutraceutical product. In certain embodiments, the composition is formulated for rectal delivery. In certain embodiments, the composition is formulated as a suppository or as an enema. In certain embodiments, the composition is formulated as a gel, a paste or an ointment. In other embodiments, the composition is formulated as a liquid, semi liquid or a suspension. In certain embodiments, the composition is formulated as a tooth paste or as an oral rinse.

The delivery of the scAb-expressing bacteria of the present invention may be made according to any method known in the art. According to some embodiments, the bacteria is encapsulated to allow sustained release and/or to improve the delivery to the treated area, for example to a specific area of the gastrointestinal system. Methods for encapsulation are known to a person skilled in the art, and are disclosed, for example, in EP0450176.

In certain embodiments, any one of the compositions described above is for use in a method of inhibiting TLR signaling in a leukocyte or in an epithelial cell. The term “inhibiting” as used herein means to prevent, decrease, limit, or block a TLR-related signal cascade. In certain embodiments, any one of the compositions described above is for use in a method of forming or increasing an immune response in a subject towards a pathogen. In certain embodiments, the pathogen comprises or secretes a ligand which activates a TLR. In certain embodiments, the pathogen is a bacterial pathogen. In certain embodiments, the bacterial pathogen is of the genus Porphyromonas. In certain embodiments, the bacterial pathogen is of the species Porphyromonas gingivalis. In certain embodiments, the transgenic bacterium is capable of expressing and secreting a scAb or an antigen-binding fragment thereof directed to TLR2. In certain embodiments, the transgenic bacterium is of the genus Lactobacillus.

In certain embodiments, any one of the compositions described above is for use in a method of preventing or ameliorating inflammation in a subject. In certain embodiments, the inflammation is associated with an oral disease or disorder. In certain embodiments, the inflammation is associated with a gastrointestinal disease or disorder. In certain embodiments, the inflammation is associated with proctitis. In certain embodiments, the inflammation is associated with radiation-induced proctitis. In certain embodiments, the inflammation is associated with inflammatory bowel disease (IBD). In certain embodiments, the inflammation is associated with Crohn's disease. In certain embodiments, the inflammation is associated with ulcerative colitis. In certain embodiments, the inflammation is associated with an endogenous ligand. In certain embodiments, the inflammation is associated with an exogenous ligand. In certain embodiments, the inflammation is associated with a ligand selected from the group consisting of bacterial lipoprotein and peptidoglycans, double stranded RNA, lipopolysaccharides, bacterial flagella, bacterial and viral single stranded RNA, CpG DNA, profilin from Toxoplasma gondii, a Damage-associated molecular patterns (DAMPs) biomolecule, and any combination thereof. Each possibility represents a separate embodiment of the invention. In certain embodiments, the inflammation is associated with an endogenous ligand selected from the group consisting of bacterial peptidoglycans, bacterial lipopolysaccharides, and a combination thereof.

The term “preventing” according to the present invention, includes prevention of recurrence, for example of an infection (protection of the host against future challenges with the pathogen), inflammation or tumor proliferation or metastasis spread.

In certain embodiments, any one of the compositions described above is for use in reducing secretion of inflammatory cytokines, for example of TNFα, by a mammalian cell. In certain embodiments, any one of the compositions described above is for use in increasing phagocytosis of a pathogen by a mammalian cell. In certain embodiments, the mammalian cell is a leukocyte selected from the group consisting of a macrophage, a neutrophil, a dendritic cell, a mast cell, a T cell, and a fibroblast. In certain embodiments, the mammalian cell is a macrophage. In certain embodiments, the method comprises inhibiting the response of a mammalian cell to stimulation by a ligand which activates a TLR.

In certain embodiments, any one of the compositions described above is for use in a method of preventing or ameliorating a gastrointestinal cancer in a subject. According to some embodiments, the gastrointestinal cancer is colon cancer.

In certain embodiments of any one of the compositions described above, the method comprises applying the composition to a mucosal surface of a subject. In certain embodiments, the composition is formulated such that it does not spread beyond the mucosal surface on which it was applied.

In certain embodiments, any one of the compositions described above comprises 10⁶ to 10¹² c.f.u. of the transgenic bacterium.

The present invention further provides, in another aspect method of inhibiting TLR signaling in a leukocyte or in an epithelial cell in a subject in need thereof, comprising administering any one of the compositions described above to the subject.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

The following examples are presented in order to more fully illustrate some embodiments of the invention. They should, in no way be construed, however, as limiting the broad scope of the invention.

### EXAMPLES

### Example 1. The Inflammatory Response to Infection with P. gingivalis is Driven by a TLR2-PI3K Pathway

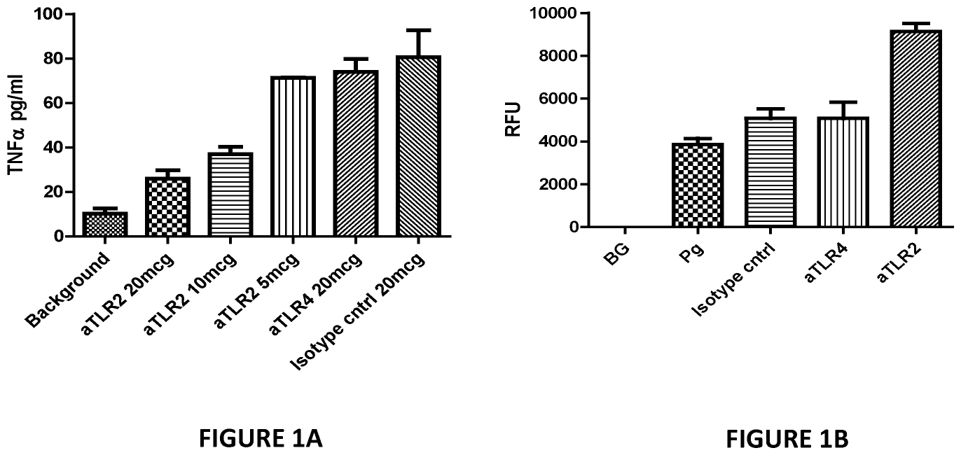

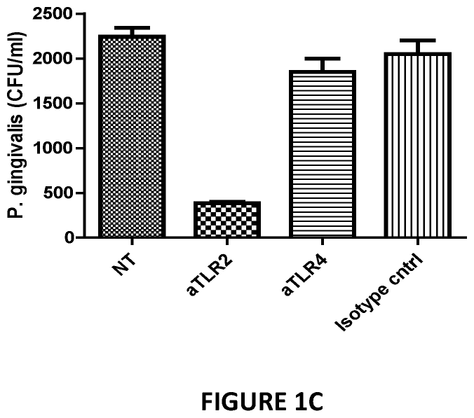

The involvement of TLR2 and TLR4 in the macrophage response to P. gingivalis was analyzed by applying inhibitory antibodies to TLR2 (T2.5) and TLR4 (1A6). These studies were performed on primary peritoneal and bone marrow-derived macrophages, as well as on murine and human macrophage cell lines, in order to probe the robustness of the involvement of TLR2 in the response to infection. Macrophages were infected with P. gingivalis at an multiplicity-of-infection (MOI) of 10, and TLRs were blocked by pre-incubation with the antibodies at increasing concentration. TLR2 inhibition reduced TNFα production in response to infection with P. gingivalis in a dose dependent manner, whereas inhibition of TLR4 had no effect as shown in FIG. 1A.

These results are consistent with the effect of TLR2 inhibition on the neutrophil response to infection and with a previous report that showed decreased cytokine production in response to infection in-vivo in Tlr2−/− mice (Burns E. et al., J. Immunol., 2006, 177(12):8296-8300). Using this in-vitro model it was next tested if blocking macrophage TLR2 would affect phagocytosis. A fluorescence plate-based assay was established to measure macrophage phagocytosis. In the assay, P. gingivalis was labelled with fluorescein isothiocyanate (FITC), and phagocytosis was monitored after washing away extracellular bacteria and quenching bacteria attached to the macrophage surface using Trypan blue. TLR2 inhibition resulted in a significant increase in macrophage phagocytosis of P. gingivalis, whereas blocking TLR4 did not affect phagocytosis as depicted in FIG. 1B.

To determine the fate of internalized bacteria when TLR2 was blocked, an intracellular survival assay was utilized. Bone marrow macrophages were infected with P. gingivalis (in the presence or absence of TLR blocking antibodies), extracellular bacteria were washed away, and cells were then treated with antibiotics to kill remaining surface attached bacteria. Macrophages were incubated for a further one hour and then lysed with water. Lysates were plated in serial dilution on blood agar plates and c.f.u. of P. gingivalis were enumerated. Blocking TLR2, but not TLR4, led to a significant increase in bactericidal activity (FIG. 1C). Therefore, together with lowering cytokine production, blocking TLR2 enhances both macrophage phagocytosis and bactericidal activity. The dominant role of TLR2 in the inflammatory response and in evasion from bactericidal activity was consistent when the assays were repeated using murine macrophages derived from other tissues (e.g. peritoneum), and when human macrophages were challenged with P. gingivalis (data not shown).

### Example 2. Anti-TLR2, Anti-TLR4 and Control scAb Cloning to Lactobacillus Expression Vector, Lactobacillus Transformation, and Expression Studies

The coding regions of the anti TLR2 and TLR4 light and heavy chain variable regions were sub-cloned into Lactobacillus expression vectors.

The mammalian secretion signals were removed from both the VL and VH domains, the constant-domains of which were determined (T2.5, that recognizes both mouse and human-TLR2) or received from outside (an anti-mouse-TLR4 mAb and an anti-human-TLR4 mAb). Expression plasmids differing in promoter sequence with one containing a constitutive promoter and the others containing sugar-inducible promoters were procured, however given the difficulties encountered in the sub-cloning, the plasmid, pTRK882 (Duong T. et al., Microbial biotechnology, 2011, 4(3):357-367), was used. The five Lactobacillus codon-optimized sequences were cloned to pTRK882 and amplified in E. coli. All plasmids contain the ermC gene conferring erythromycin resistance. Competent Lactobacillus was prepared and transformed by electroporation using standard procedures for transformation.

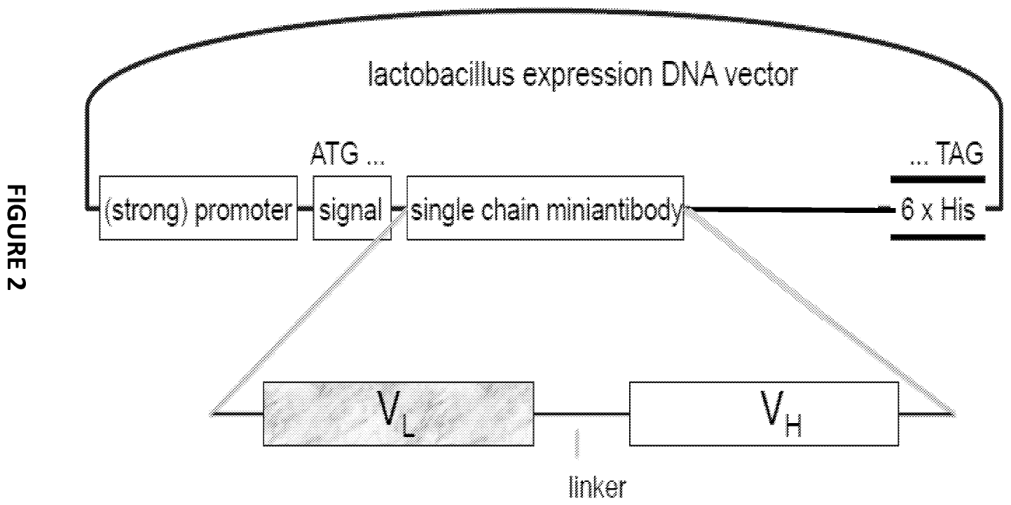

Lactobacillus transformation was successful for the empty vector, and the vectors carrying scAbs to human TLR4, murine TLR4, and the anti-TLR2 that recognizes both mouse and human TLR2 (FIG. 3).

Despite multiple transformation attempts and despite modifications to the transformation protocol, it was not possible to obtain transformants of the anti-TLR2 antibody when an additional domain, the IgG1 Fc domain, was fused to the C-terminus. Since the promoter and regulatory elements, as well as the ermC resistance gene were identical for these constructs, it was concluded that the size of the protein was too large for expression in Lactobacillus. In the functional experiments, Lactobacillus transformed with the empty plasmid was used as a control. Although this is a less ideal control than an irrelevant scAb control, as will be shown below, the anti-TLR4 scAb expressing-Lactobacillus also serves as a control for the specificity of TLR2 in the response to P. gingivalis.

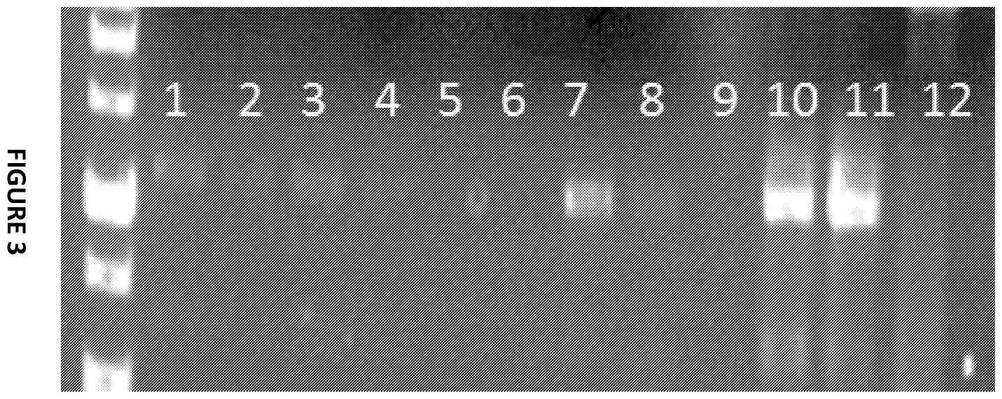

As shown in FIG. 2, the constructs all carried a 6×His tag for detection of the secreted protein. The Lactobacillus transformants were next cultured to varying concentrations, and the bacterial supernatants were tested for scAb content by anti-His tag immunoblot. As shown in FIG. 4, Lactobacillus transformants that secrete proteins of the predicted MW were obtained, and no His-tagged protein is detected in the supernatants of EV-transformed bacteria.

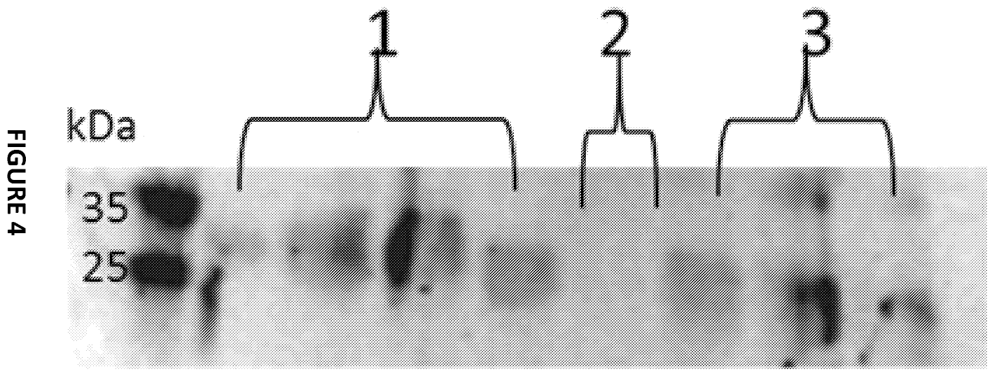

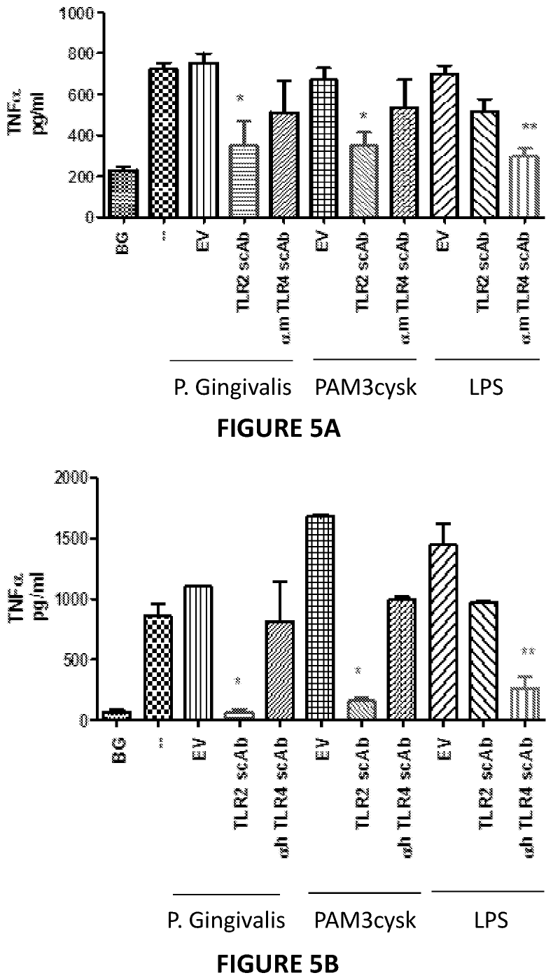

The scAbs were next tested for their ability to functionally and specifically block TLR responses. Protein concentrates from the Lactobacillus transformant supernatants were added to macrophages prior to challenge with a ligand of TLR2 (PAM3CSK4) or a ligand of TLR4 (E. coli LPS), or with P. gingivalis (a TLR2-stimulant, as shown above). As a control, proteins concentrated from the empty vector (EV) transformants were added to the cells, and they did not inhibit TLR-driven responses (FIG. 5). In contrast, the anti-TLR2 scAb blocked the response to PAM3CSK4, and not LPS, in both murine and human macrophages, the anti-human TLR4 scAb specifically blocked the response to LPS by human macrophages, and the anti-mouse TLR4 scAb blocked the response to LPS in the murine macrophages (FIG. 5). As with the full length mAbs, the response to P. gingivalis was specifically inhibited by the scAb against TLR2.

The results presented above support the strong ability of scAbs produced by non-pathogenic bacteria, such as Lactobacillus, to specifically block murine and human TLR2 and/or TLR4. This indicates that bacteria can secrete functional blocking mini-bodies against surface-expressed TLRs, such as TLR 2 and TLR 4. As a therapeutic agent, engineered probiotic bacteria have the potential to modulate host TLR2/4-driven pathogenic infections and mucosal inflammatory damage.

### Example 3. In-Vivo Activity of Transformed Lactobacillus

The Lactobacillus transformants were tested for their ability to block TLR2 in-vivo. In this model, two titanium chambers are inserted subcutaneously to each mouse seven days prior to the experiment. The Lactobacillus transformants were then injected to the chambers 24 hours prior to challenge with live P. gingivalis. Exudates from the chambers were obtained at 2 hours and 24 hours post challenge (each chamber is drained once), and the fluid was tested for inflammatory cytokines and plated to determine the number of live colonies of P. gingivalis.

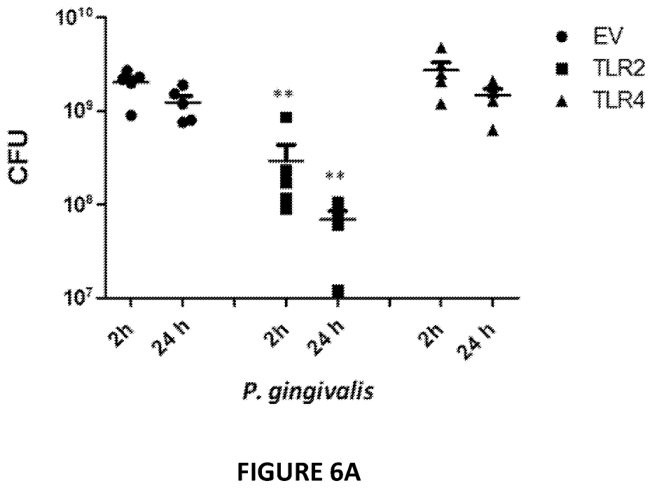

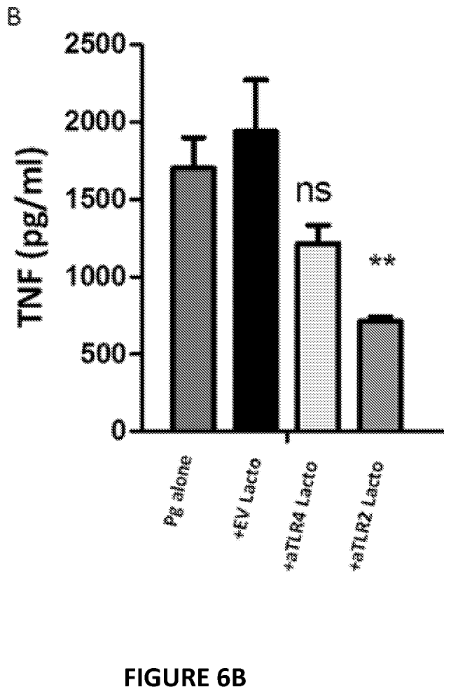

Mice received one of three different types of transformed lactobacilli prior to challenge with P. gingivalis: lactobacilli transformed with an empty vector (EV), lactobacilli transformed to secrete anti-TLR2 scAb, or lactobacilli transformed with to secrete anti-murine TLR4 scAb. P. gingivalis' survival in-vivo was identical between the mice that received the empty-vector-transformed or the anti-TLR4-secreting lactobacilli, however mice that received the lactobacilli secreting anti-TLR2 scAb demonstrated significantly greater clearance of P. gingivalis (FIG. 6A), associated with reduced TNF production by these mice in response to P. gingivalis challenge (FIG. 6B).

The results demonstrate that P. gingivalis is specifically sensitive to anti-TLR2 scAb secreted from transformed lactobacilli, and that this effect is linked to reduced TNFα levels.

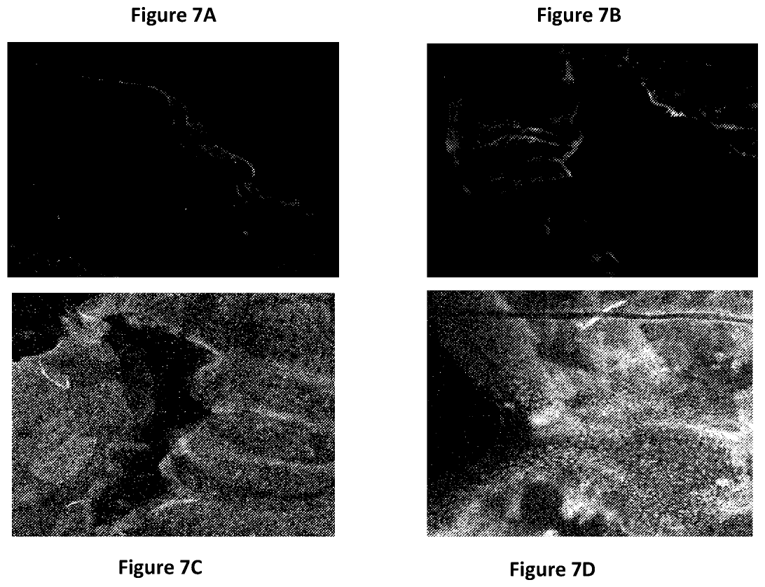

The ability of Lactobacillus transformants to secrete functional single chain antibody (scAb) in the oral cavity, and the ability of the scAb to bind to oral tissue that express TLR2 was next assessed. Mice were administered by one oral lavage the following Lactobacillus transformants: EV (empty vector transformed, negative control), scahTLR4 (single chain antibody against human TLR4, negative control), and scaTLR2 (single chain antibody against TLR2). After 24 h the mice were sacrificed, Lactobacillus persistence was assessed by culturing viable bacteria and enumerating CFU, and the maxillary gingival tissues were processed for whole mounting and stained with an antibody to the His-tag present in the single chain antibodies Similar Lactobacillus CFU were recovered in all three groups. Anti-His staining revealed strong positive staining to epithelial and non-epithelial cells of the oral tissue only for the scaTLR2-secreting transformants (FIG. 7C-D). In the control groups there was no detection of His-positive staining (FIG. 7A-B). Therefore, scaTLR2 is secreted by the transformed Lactobacilli in vivo, and avidly binds to oral mucosal tissue.

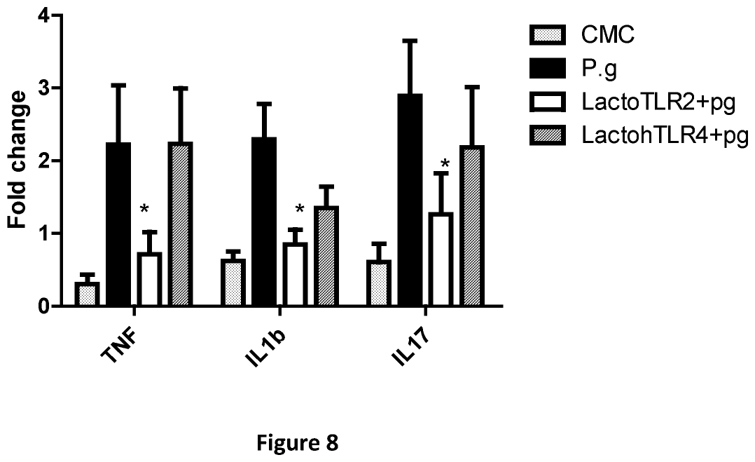

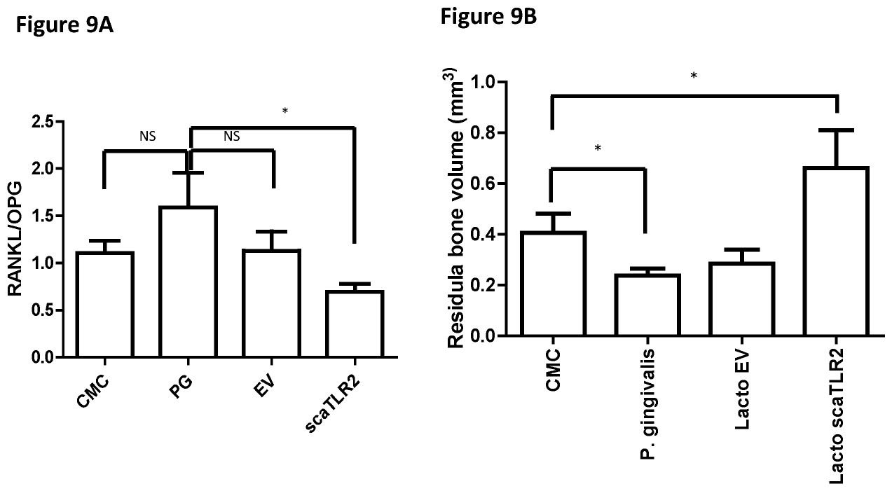

Next, the ability of the scaTLR2-secreting Lactobacillus to prevent oral tissue damage induced by the periodontal pathogen P. gingivalis was tested. Mice were administered Lactobacilli every other day intermittently with the administration of P. gingivalis for a total of three times. All bacterial administrations were done mixed with carboxy-methyl cellulose (CMC) and administered to the oral cavity by lavage. The following Lactobacillus transformants were tested: scahTLR4 (single chain antibody against human TLR4, negative control), and scaTLR2 (single chain antibody against TLR2). 72 hours after the last administration of P. gingivalis, groups of 5 mice were sacrificed and the gene expression level in the gingival tissue of Tnf, Il1b, and Il117 was determined. The expression level of all inflammatory cytokines was significantly reduced by treatment with Lactobacilli secreting scaTLR2 (FIG. 8). Using a similar treatment protocol, groups of mice were treated with Lactobacilli EV (empty vector transformed, negative control) vs. Lactobacilli secreting scaTLR2 (single chain antibody against TLR2) intermittently with administration of P. gingivalis and then followed for six weeks at which time they were sacrificed, and bone volume surrounding the teeth was determined by microCT. Remarkably, in addition to preventing alveolar bone resorption induced by P. gingivalis infection, Lactobacilli secreting scaTLR2 significantly increased the bone volume compared to non-infected controls (FIG. 9B). Induction of RANKL relative to OPG, a marker of osteoclast activation, was also significantly prevented by administering the anti-TLR2 secreting lactobacilli (FIG. 9A). Thus, the anti-TLR2 secreting lactobacilli recapitulate the phenotype of TLR2-deficient mice—these mice are resistant to P. gingivalis-induced periodontitis and studies have shown greater alveolar bone volume compared to wild-type mice.

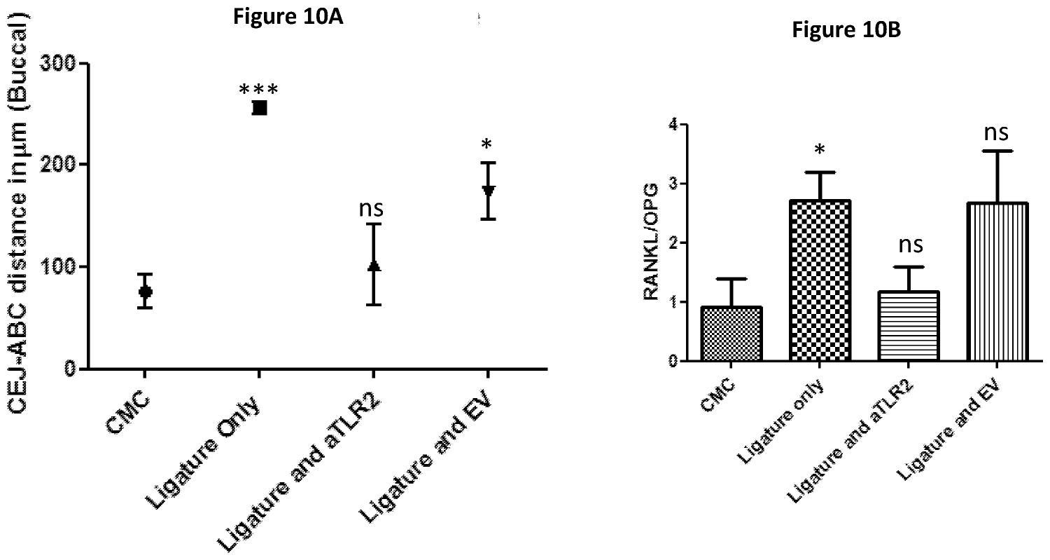

This finding led to test the effect of Lactobacilli secreting scaTLR2 in the ligature-induced model of periodontitis (Abe T., Hajishengallis G. 2013, J Immunol Methods, 394:49-54). In this model, a ligature is placed around the second maxillary molar leading to rapid inflammation and bone resorption. Importantly, there is no administration of P. gingivalis or any other exogenous periodontal pathogen in this model. Rather, the disruption of tissue homeostasis due to the ligature enables the endogenous microflora to induce inflammation, osteoclast activation, and bone resorption. Lactobacilli EV (empty vector transformed, negative control) vs. Lactobacilli secreting scaTLR2 (single chain antibody against TLR2) were administered by oral lavage daily following ligature placement for four days and mice were sacrificed on day 7 following ligature placement. As expected, ligature placement led to highly significant bone resorption (FIG. 10A) and increased RANKL/OPG ratio (FIG. 10B). Remarkably, Lactobacilli secreting scaTLR2 completely prevented bone resorption due to the ligature, and blocked the increase in RANKL/OPG. Administration of Lactobacilli EV had a mild effect by reducing the extent of bone resorption although it was still significant compared to the non-ligated control group. Thus, the administration of lactobacilli secreting scaTLR2 demonstrates a dual protective effect, attributable to the vehicle (the lactobacilli), and to the single chain antibody.

### Example 4. In-Vivo Activity of Transformed Lactobacillus in DSS-Induced Colitis

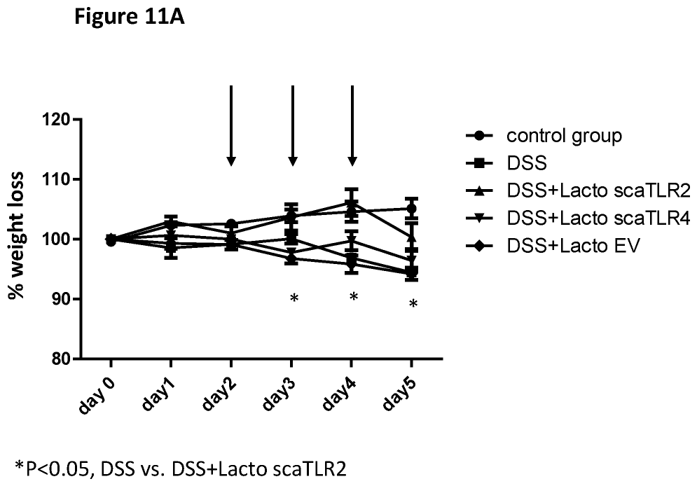

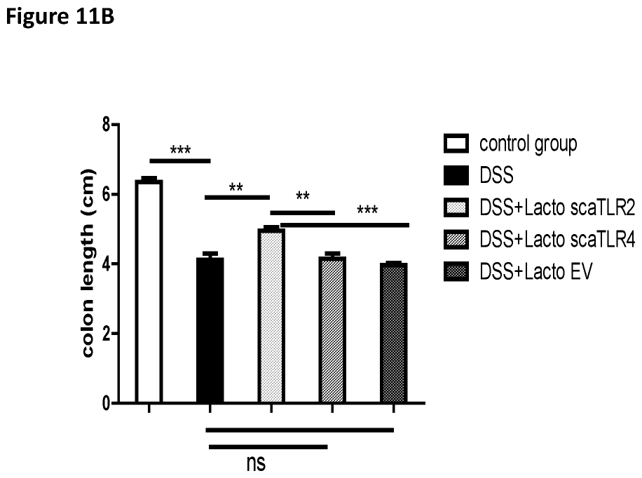

Next, the ability of the scaTLR2-secreting and scaTLR4-secreting lactobacilli vs Lactobacilli EV to treat colonic inflammation induced by administration of Dextran sodium sulfate (DSS) was tested. DSS was administered continuously in the drinking water throughout the experiment, and on the third day mice were administered Lactobacilli once daily for three days. Mice were weighed daily and sacrificed on the sixth day (one day after the final Lactobacilli administration). Following sacrifice, the colonic length of each mouse was determined. As shown in FIG. 11A, administration of Lactobacilli secreting scaTLR2, but not lactobacilli secreting scaTLR4 (murine) or Lactobacilli EV, effectively protected mice from weight loss due to colonic inflammation induced by DSS. The effect on colon length at sacrifice confirmed the therapeutic effect of lactobacilli secreting scaTLR2. DSS treated mice showed an expected shortening of the colon due to severe inflammation. Colon length in the scaTLR4 and Lactobacilli EV groups was similar to the DSS group, however the colon length in the scaTLR2 treated group was significantly longer, reflecting reduced inflammation.

While the foregoing is directed to embodiments of the present invention, other and further embodiments of the invention may be devised without departing from the basic scope thereof, and the scope thereof is determined by the claims that follow.

1-65. (canceled)

66. A transgenic bacterium capable of expressing and secreting an exogenous single chain antibody (scAb) or an antigen-binding fragment thereof that specifically recognizes a mammalian Toll-like receptor (TLR) selected from TLR2 and TLR4.

67. The transgenic bacterium of claim 66, wherein the scAb is expressed in the transgenic bacterium from an exogenous expression cassette comprising a transcribable polynucleotide operably linked to an expression control sequence, wherein the transcribable polynucleotide encodes a scAb comprising an amino acid sequence selected from SEQ ID NO: 1 and SEQ ID NO: 2.

68. The transgenic bacterium of claim 66, wherein the transgenic bacterium is capable of reproduction on a mucosal surface selected from the group consisting of: oral mucosa, nasal mucosa, gastrointestinal mucosa, vaginal mucosa and urinary bladder mucosa, or on the skin of a mammalian subject.

69. The transgenic bacterium of claim 66, wherein the bacterium constantly expresses and secretes the scAb or the antigen-binding fragment thereof.

70. The transgenic bacterium of claim 66, wherein the transgenic bacterium is incapable of reproduction on a mucosal surface or in the body of a subject because of a biocontainment strategy applied, wherein the viability or reproductivity of the transgenic bacterium in a human host is dependent on a compound not found naturally in healthy humans.

71. The transgenic bacterium of claim 66, wherein the transgenic bacterium is selected from the group consisting of: a probiotic bacterium, a commensal bacterium, a mutualistic bacterium and a bacterium that is non-pathogenic in humans.

72. The transgenic bacterium of claim 71, wherein the transgenic bacterium is of the order Lactobacillales.

73. The transgenic bacterium of claim 66, wherein the scAb or the antigen-binding fragment thereof is directed to an extracellular domain of a human TLR2 or TLR4 expressed by a cell selected from the group consisting of a macrophage, a neutrophil, a dendritic cell, a mast cell, a T cell, a fibroblast and an epithelial cell.

74. The transgenic bacterium of claim 66, wherein the scAb that specifically recognizes TLR2 comprises the six complementarity determining regions (CDR) sequences set forth in SEQ ID NOs: 3 to 8.

75. The transgenic bacterium of claim 66, wherein the scAb that specifically recognizes TLR2 comprises the amino-acid sequence set forth in SEQ ID NO: 1.

76. The transgenic bacterium of claim 66, wherein the scAb that specifically recognizes TLR4 comprises the six CDR sequences set forth in SEQ ID NOs: 9 to 14.

77. The transgenic bacterium of claim 66, wherein the scAb that specifically recognizes TLR4 comprises the amino-acid sequence set forth in SEQ ID NO: 2.

78. The transgenic bacterium of claim 66, wherein the scAb is composed of a heavy-chain variable region and light-chain variable region connected directly or through a spacer or a linker.

79. A transformed strain of bacteria, comprising a polynucleotide sequence that encodes a scAb selected from SEQ ID NO: 1 and SEQ ID NO: 2, or a binding fragment thereof that specifically recognizes a mammalian TLR selected from TLR2 and TLR4.

80. A composition comprising at least one transgenic bacterium according to claim 66.

81. The composition of claim 80, comprising at least two different bacteria, wherein each bacterium expresses and secretes a different scAb or an antigen-binding fragment thereof directed to a different TLR.

82. A method of inhibiting TLR signaling in a mammalian cell in a subject in need thereof, comprising the step of administering the composition of claim 80 to the subject, thereby inhibiting TLR signaling.

83. A method of treating a pathogenic infection caused by a bacterial pathogen in a subject in need thereof, comprising the step of administering a composition according to claim 80 to the subject, thereby treating the pathogenic infection.

84. A method of treating an inflammation or a cancer associated with TLR activation in a subject in need thereof, comprising the step of administering a composition according to claim 80 to the subject, thereby treating the inflammation or the cancer,
wherein the inflammation is associated with:
an oral disease or disorder;
a gastrointestinal (GI) disease or disorder selected from an inflammatory bowel disease (IBD), Crohn's disease, and ulcerative colitis;
proctitis; or
a genitourinary (GU) disease or disorder, and
wherein the cancer is:
a cancer of the GI system selected from colon cancer, gastric cancer, esophageal cancer, and adenocarcinoma; or
a cancer of the GU system selected from urinary cancer, bladder cancer and prostate cancer.

85. A kit comprising a composition according to claim 80, a separate container comprising an agent capable of activating biocontainment strategy, and optional instructions of use.


SEQUENCE LISTING (15 sequences)

SEQ ID NO: 1 - protein, 275 aa, Artificial Sequence
  single chain antibody
MKQTKKFWLLLILLVSTLGMALEQTQTVNAQVQLQQSGPELVNPGASVKLSCKASGFTFTTYGINWVKQGPGQGLEWIGWIYPRDGSTNFNENFKDKAALTVDTSSSTAYMELHSLTSEDSAVYFCARLTGGTFLDYWGQGTTVTVSSGGGGSGGGGSGGGGSDIELTQSPASLAVSLGQRATISCRASESVEYYGTSLMQWYQQKPGQPPKLLIFGASNVESGVPVRFSGSGSGTDFSLNIHPVEEDDIVMYFCQQSRKLPWTFGGGTKLEIKR

SEQ ID NO: 2 - protein, 274 aa, Artificial Sequence
  single chain antibody
MKQTKKFWLLLILLVSTLGMALEQTQTVNAQVTLKESGPGILQPSQTLSLTCSFSGFSLTTYNIGVGWIRQPSGKGLEWLAHIWWNDNIYYNTVLKSRLTFSKDTSNNQVFLKIASVDIADTATYYCIRMAEGRYDAMDYWGQGTSVTVSSGGGGSGGGGSGGGGSAIQMTQSTSSLSASLGDRVTINCRASQDITNYLNWYQQKPDGTVRLLIYYTSKLHSGAPSRFSGRGSGTDYSLTISNLEQEDIATYFCQQGNTFPWTFGGGTKLEIKR

SEQ ID NO: 3 - protein, 5 aa, Artificial Sequence
  CDR
TYGIN

SEQ ID NO: 4 - protein, 17 aa, Artificial Sequence
  CDR
WIYPRDGSTNFNENFKD

SEQ ID NO: 5 - protein, 9 aa, Artificial Sequence
  CDR
LTGGTFLDY

SEQ ID NO: 6 - protein, 15 aa, Artificial Sequence
  CDR
RASESVEYYGTSLMQ

SEQ ID NO: 7 - protein, 7 aa, Artificial Sequence
  CDR
GASNVES

SEQ ID NO: 8 - protein, 9 aa, Artificial Sequence
  CDR
QQSRKLPWT

SEQ ID NO: 9 - protein, 6 aa, Artificial Sequence
  CDR
GFSLTT

SEQ ID NO: 10 - protein, 8 aa, Artificial Sequence
  CDR
HIWWNDNI

SEQ ID NO: 11 - protein, 9 aa, Artificial Sequence
  CDR
EGRYDAMDY

SEQ ID NO: 12 - protein, 6 aa, Artificial Sequence
  CDR
QDITNY

SEQ ID NO: 13 - protein, 7 aa, Artificial Sequence
  CDR
YTSKLHS

SEQ ID NO: 14 - protein, 9 aa, Artificial Sequence
  CDR
QQGNTFPWT

SEQ ID NO: 15 - protein, 15 aa, Artificial Sequence
  Linker
GGGGSGGGGSGGGGS